(19) United States
(12) Patent Application Publication
(10) Pub. No.: US 2009/0029355 A1
(43) Pub. Date: Jan. 29, 2009

(54) Abnormalities of Phosphatase 2A (PP2A) for Diagnosis and Treatment of Alzheimer's Disease

(76) Inventors: Wei-Qin Zhao, Gwynedd, PA (US); Daniel L. Alkon, Bethesda, MD (US)

Correspondence Address: MILBANK, TWEED, HADLEY & MCCLOY LLP, INTERNATIONAL SQUARE BUILDING, 1850 K STRET, N.W., SUITE 1100, WASHINGTON, DC, 20006, US

(21) Appl. No.: 11/660,868

(22) PCT Filed: Nov. 15, 2004
(86) PCT No.: PCT/US04/38160

§ 371 Date: Oct. 3, 2008

(51) Int. Cl. C12Q 1/68; C12Q 1/42; G01N 33/53; C07K 16/00

(52) U.S. Cl. 435/6; 435/21; 435/7.92; 530/387.1

(57) ABSTRACT

This invention relates to methods of diagnosing Alzheimer's disease and methods of screening for compounds for the treatment or prevention of Alzheimer's disease. The methods are based upon newly discovered differences in protein phosphatase 2A (PP2A) function and related molecular events in Alzheimer's disease cells compared to control cells. In one embodiment, differences in basal PP2A gene expression in Alzheimer's cells are compared to controls. In another embodiment differences in PP2A protein and enzyme activity are compared in test and control cells. In another embodiment differences in response to substances that inhibit PP2A function are compared. Still another embodiment detects differences in the subcellular distribution of phosphorylated Erk1/2, a substrate of PP2A, in normal and Alzheimer's disease cells. The detection of Alzheimer's disease-specific differences in PP2A function and related events in peripheral tissues provides the basis for highly practical and efficient tests and diagnostic test kits for the early diagnosis of Alzheimer's disease, as well as providing a biochemical basis for identifying therapeutic targets for drug development.

### FIELD OF THE INVENTION

This invention relates to methods of diagnosing Alzheimer's disease. The methods are based upon newly discovered differences in protein phosphatase 2A (PP2A) expression or function and related molecular events in cells of Alzheimer's disease patients compared to control cells. The detection of Alzheimer's disease-specific differences of PP2A expression and function in peripheral tissues provides the basis for highly practical and efficient tests for the early diagnosis of Alzheimer's disease, and for therapeutic drug development.

### BACKGROUND

Dysfunction of protein phosphorylation, particularly that due to an impaired phosphatase pathway, has been implicated in the molecular pathology of Alzheimer's disease (AD). One of the major examples of such abnormality is hyperphosphorylation of the microtubule-associated tau protein that constitutes neurofibrillary tangles (NFT), which represents one of the most prominent lesions in the brain of Alzheimer's disease (Cummings et al., 1998; Jellinger and Bancher, 1998). In a normal neuron, tau binds to tubulin and thereby participates in microtubule assembly. Phosphorylation of tau reduces the microtubule binding leading to destabilization of the neuronal cytoskeleton (Lee, 1995; Billingskey and Kincaid, 1997). When tau is hyperphosphorylated, it loses the ability to bind microtubules and is believed to self-assemble into paired helical filaments (PHF), an indication of aberrant cytoskeletal protein processes (Lee, 1995; Billingsley and Kincaid, 1997; Saito et al., 1995; Mandelkow et al., 1995).

In the search for mechanisms that underlie AD-associated molecular abnormalities, much attention has been focused on the protein kinases and phosphatases that regulate tau phosphorylation. Several protein kinases, including glycogen synthase kinase-3 (GSK-3) and mitogen-activated protein (MAP) kinase have been found to phosphorylate tau. Normal activity of MAP kinase controls cell proliferation and differentiation (Force and Bonventre, 1998; Roovers and Assoian, 2000), and plays an important role in brain functions such as learning and memory (Valijent et al., 2001; Sweatt, 2001; Zhao et al., 1999). On the other hand, abnormally sustained MAP kinase activation can be harmful by causing tau overphosphorylation and neuronal apoptosis (Guise et al., 2001). Sustained activation of the extracellular signal-regulated kinase (Erk), a member of the MAP kinase family, was induced by β-amyloid in the rodent hyppocampal neurons (Rapoport and Ferreira, 2000; Dineley et al., 2001), which in turn caused increases in tau phosphorylation, neurite degeneration, and neuronal death (Rapoport and Ferreira, 2000). In addition, prolonged Erk1/2 phosphorylation is found in AD fibroblasts induced by bradykinin, a potent inflammation mediator (Zhao et al., 2002), and an association between activated Erk2 and neurofibrillary tangles has been demonstrated in the human brain (Knowles et al., 1999).

Dysfunction of phosphatase activities can also contribute critically to aberrant protein phosphorylation in AD. Among the four major types of serine/threonine protein phosphatase (phosphatase-1, 2A, 2B, and 2C) in organic cells, phosphatase 2A (PP2A) isoforms are abundantly expressed in the brain and targets to specific localization of intracellular protein such as neurofilaments (Saito et al., 1995; Janssens and Goris, 2001) and microtubule-associated proteins (Mandelkow et al., 1995; Janssens and Goris, 2001). By binding to and regulating phosphorylation of microtubule proteins such as tau and MAP2, PP2A plays an important role in maintaining microtubule stability (Mandelkow et al., 1995). In addition, PP2A has been shown to dephosphorylate specific sites of hyperphosphorylated tau in vitro and in vivo (Goedert et al., 1992; Wang et al., 1995; Gong et al., 2000; Planel et al., 2001). For example, it dephosphorylates hyperphosphorylated tau in the already formed PHFs, resulting in dephosphorylated tau detached from PHFs that become accessible to proteolysis (Wang et al., 1995). A healthy PP2A system is not only essential for maintenance of cytoskeletal stability in normal cells, but is also vital for correcting abnormally enhanced protein phosphorylation under pathological conditions such as cellular stress and high calcium toxicity. In the final stage of AD, PP2A gene expression and activity are markedly reduced (Gong et al., 1995; Vogelsberg-Ragaglia et al., 2001). In another study, expression of a mutant form of PP2A in mouse brain caused a marked decrease in PP2A activity and induced AD-like hyperphosphorylation of tau at specific serine/threonine residues (Kins et al., 2001).

PP2A has been found to be responsible for inactivation of MAP kinase in several types of cells (Alessi et al., 1995; Braconi Quintaje et al., 1996; Chung and Brautigan, 1999), indicating that PP2A may act as a negative regulator of Erk2 activity. Recent studies showed that the inactivation of MAP kinase by PP2A was specifically regulated by the R2/B regulatory subunit of PP2A (Silverstein et al., 2002). We have previously shown that a bradykinin-stimulated Erk1/2 phosphorylation is abnormally prolonged in AD cells (Zhao et al., 2002).

A prominent pathological hallmark in the brain of relatively early stages of Alzheimer's disease (AD) is the intraneurofibrillary lesions referred to as neurofibrillary tangles (NFTs). In AD, 95% of NFT lesions are formed from paired helical filaments (PHFs). The major component of PHFs is hyperphosphorylated microtubule-associated protein tau, which causes instability of cytoskeletal proteins. Phosphatase 2A (PP2A) is the major enzyme responsible for dephosphorylation of tau. By regulating dephosphorylation of tau, PP2A participates in maintenance of normal microtubule stability in normal cells and reduces aberrantly phosphorylated tau in already formed PHFs in pathological conditions. PP2A is also one of the two phosphatases that dephosphorylate Erk1/2, a member of the MAP kinase family. By timely dephosphorylation of Erk1/2 after mitogenic or inflammatory stimulations, PP2A plays a primary role in protecting cells from apoptosis.

The present invention is based, in part, on the findings that impaired PP2A function is implicated as one of the molecular mechanisms underlying AD pathogenesis. Because direct access to neurons in the brains of living human beings is impossible, early diagnosis of AD is extremely difficult. By testing AD-specific abnormalities of PP2A and related molecular events, including Erk1/2 phosphorylation and distribution in skin cells of AD patients, the present invention is directed, in certain embodiments, to highly practical and efficient tests for early diagnosis of AD as well as diagnostic test kits and methods for therapeutic drug development.

### SUMMARY OF THE INVENTION

In one embodiment, the present invention provides methods for the diagnosis of Alzheimer's disease using human cells. The invention is based upon the discovery by the inventors of differences in PP2A expression and function and related molecular events in Alzheimer's disease cells compared to control cells. It is contemplated that any or all of the diagnostic methods of the present invention may be used in combination with any or all of the diagnostic methods described in WO 02/067764, which is herein incorporated by reference in its entirety. In one embodiment, the methods of diagnosing Alzheimer's disease based on abnormally enhanced phosphorylation of extracellular signal-regulated kinase type 1 or 2 (Erk1/2) after stimulation with an agent such as bradykinin, and the related methods of diagnosing Alzheimer's disease described in WO 02/067764, are used in any combination with the methods for diagnosing Alzheimer's disease disclosed herein.

The present invention provides a number of criteria relating to PP2A which improve the specificity and efficiency of diagnostic tests for the detection of Alzheimer's disease. Detection of Alzheimer's disease-specific differences of PP2A function in peripheral tissues also provides a biochemical basis for identifying therapeutic targets for drug development for the treatment of Alzheimer's disease.

In one aspect, the invention relates to a method of diagnosing Alzheimer's disease by detecting differences in the levels of PP2A gene expression in Alzheimer's disease cells compared to control cells. This embodiment is based upon the discovery by the inventors that fibroblasts from patients of both familial and sporadic AD present significantly higher basal levels of PP2A gene expression compared to normal cells from age-matched individuals. Preferably, detection of PP2A gene expression is performed using reverse transcription quantitative polymerase chain reaction. In a preferred embodiment, mRNA encoding PP2A is quantified in test cells and compared to levels measured in non-Alzheimer's control cells.

In another aspect, the invention relates to methods of diagnosing Alzheimer's disease by detecting differences in PP2A gene expression in test and control cells in response to compounds that stimulate phosphorylation of a protein such as Erk1/2, which is part of a signal transduction cascade that subsequently activates PP2A including gene expression of PP2A. Lack of increased PP2A expression in stimulated cells compared to unstimulated cells indicates the presence of Alzheimer's disease. Because PP2A directly dephosphorylates Erk1/2, Erk1/2 is a PP2A substrate. PP2A also dephosphorylates many other proteins. On the other hand, Erk1/2 can also be dephosphorylated by other phosphatases in addition to PP2A. However, abnormal PP2A activity and gene expression are specifically associated with enhanced Erk1/2 phosphorylation in Alzheimer's fibroblast cells in response to bradykinin stimulation. In a specific embodiment the stimulator agent is bradykinin. Other possible stimulator agents include, but are not limited to, insulin, phobol esters, lysophosphatidylcholine, lipopolysaccharide, anthracycline dannorubicin and vanadyl sulfate, which all activate MAP kinase phosphorylation via an upstream signaling pathway. This embodiment is based upon the discovery that normal cells respond to stimulation by compounds such as bradykinin by upregulating PP2A gene expression. In contrast, this normal response is lacking in Alzheimer's disease cells.

In yet another aspect, the invention relates to methods of diagnosing Alzheimer's disease by detecting differences in PP2A protein levels and/or enzymatic activities in Alzheimer's disease cells compared to control cells, where a reduction in PP2A protein levels and/or enzymatic activity indicates the presence of Alzheimer's disease. This embodiment is based upon the discovery by the inventors that both PP2A protein levels and PP2A enzymatic activity are significantly reduced in Alzheimer's disease cells compared to normal cells.

In another aspect, the invention relates to methods of diagnosing Alzheimer's disease by assessing the response of cells to stimulation by agents such as bradykinin in the presence of a PP2A inhibitor. In a specific embodiment, the PP2A inhibitor is okadiac acid. This embodiment is based upon the discovery that normal cells treated with bradykinin in the presence of okadiac acid prolonged Erk1/2 phosphorylation, which is otherwise returned to a basal level by about 10 minutes after bradykinin stimulation. This normal response is absent in Alzheimer's disease cells.

In another aspect, the invention relates to methods of diagnosing Alzheimer's disease in a subject by assessing the subcellular distribution of phosphorylated Erk1/2 in cells. This embodiment is based upon the discovery that phosphorylated Erk1/2 is concentrated in the nucleus of normal cells, but in Alzheimer's disease cells, phosphorylated Erk1/2 is distributed in the extranuclear area (i.e. the cytosolic compartment).

The methods described herein can be used alone or in any combination as highly specific and efficient tests for diagnosing Alzheimer's disease.

In yet a further aspect, this invention relates to methods of screening therapeutic substances for the treatment or prevention of Alzheimer's disease using the tests described herein. The screening methods are based on the discoveries made by the inventors of Alzheimer's disease-associated abnormalities in PP2A and related molecular events as further described herein.

This invention also relates to kits comprising products useful for carrying out the diagnostic methods of the invention.

The diagnostic methods and methods for treating Alzheimer's disease of the present invention are based on the following observations made for the first time by the inventors.

Fibroblasts from patients of both familial and sporadic AD present significantly high basal levels of PP2A gene expression compared to normal cells from age-matched individuals.

Normal age-matched control (AC) cells respond to BK stimulation with upregulation of PP2A gene expression. This normal response is lacking in AD cells.

Both PP2A protein levels and enzymatic activities in AD cells are significantly reduced compared to AC cells.

Treatment of AC cells with BK in the presence of okadiac acid (OA), a PP2A inhibitor, prolonged Erk1/2 phosphorylation, which is otherwise returned to a basal level by about 10 min after BK stimulation. Because the BK-stimulated Erk1/2 phosphorylation is sustained in AD cells due to inhibition of the normal dephosphorylation mechanism, application of OA has no additional effect on the extent of Erk1/2 phosphorylation. Thus, the ratio of +OA/−OA Erk1/2 phosphorylation in AC cells is significantly greater than that in AD cells.

When Erk1/2 is phosphorylated in AC cells it is concentrated in the nucleus, but in AD cells phosphorylated Erk1/2 is distributed in the extranuclear area.

All of the differences cited above between AC and AD cells form the basis for the clinical tests and diagnostic kits for Alzheimer's disease diagnosis, as well as the methods of screening compounds for treatment or prevention of Alzheimer's disease disclosed herein.

In a preferred embodiment of the invention, human skin fibroblasts are used in the tests and diagnostic assays of the invention, but blood cells might also be used. In one embodiment, cells from the same individual can be cultured in several flasks for pharmacological treatment.

In one embodiment, PP2A gene expression is examined with reverse transcription quantitative PCR (RVQ-PCR) using a Taqman® real-time PCR device with either a 384- or 96-well microplate. In certain embodiments, a reference gene that is abundantly expressed in the eukaryotic cell such as GAPDH is simultaneously amplified and used for normalization.

In one embodiment, PP2A protein levels and Erk1/2 phosphorylation are examined by Western blotting or ELISA.

In one embodiment, nuclear translocation of Erk1/2 is measured. Cells are stimulated by BK and the nuclear distribution of activated Erk1/2 is examined by either immunocytochemistry, or by determining a test ratio of phospho-Erk1/2 between the nucleus and the cytosol.

The serine/threonine phosphatase 2A (PP2A) has been implicated in the pathogenesis of Alzheimer's disease (AD) due to its important role in regulating dephosphorylation of microtubule-associated protein tau and mitogen-activated protein (MAP) kinase. The inventors have found that PP2A is responsible for dephosphorylation of the extracellular signal-regulated kinase 1/2 (Erk1/2) following its activation by BK stimulation. The inventors have also found that abnormal gene and protein expression of PP2A, as well as abnormal PP2A activity, contribute to the abnormally prolonged Erk1/2 phosphorylation in AD fibroblasts. Inhibition of PP2A with okadiac acid produces enhanced and more lasting Erk1/2 phosphorylation after BK stimulation, whereas FK506, an inhibitor of PP2B and FK-binding protein, inhibits the BK-stimulated Erk1/2 phosphorylation. Furthermore, while the phosphorylated Erk1/2 is concentrated in the nucleus of AC cells, it is mainly distributed in the extranuclear compartments of AD cells. The inventors have found that the delayed dephosphorylation of Erk1/2 in AD cells following its BK-stimulated activation is due to deficits of PP2A activity and impaired nuclear translocation of phosphorylated Erk1/2.

### BRIEF DESCRIPTION OF THE DRAWINGS

FIG. 1A-1D: Detection of PP2A and GAPDH gene expression via RTQ-PCR: Total RNA from the human fibroblast culture was extracted and the first-strand cDNA was generated as described herein. Linear plots for PP2A and GAPDH standard curves are presented in FIG. 1A and FIG. 1B. FIG. 1C shows the disassociation curve plots for different melting temperatures of PP2A and GAPDH. In FIG. 1D, the final PCR products of PP2A and GAPDH with expected sequence sizes are revealed on a TBE gel (lanes 2 and 4). No PCR products were amplified from samples that underwent reverse transcription in the absence of reverse transcriptase (lanes 1 and 3).

FIG. 2A-2B: Quantification of PP2A gene expression by RTQ-PCR: During real-time PCR, levels of PP2A and GAPDH mRNA were automatically calculated by the instrument based on the standard curve for each gene simultaneously performed on the same PCR run. The ratios of PP2A mRNA levels over GAPDH levels from each AC and AD cell line were calculated and presented in FIG. 2A. Statistical analysis using a t test indicates a significant difference in PP2A mRNA levels between AC and AD cells (P<0.01). When treated with 10 nM BK for about 10 min, an upregulation of PP2A mRNA was observed in AC but not in AD cells (FIG. 2B). A t test indicates significant treatment effects between AC and AD cells (*P=0.016).

FIG. 3A-3B: PP2A protein levels and enzymatic activities in AC and AD fibroblasts: Cell lysates from AC and AD cells were prepared as described herein. In FIG. 3A, the same sample volume from eight AC and eight AD cell lines, after being treated with SDS-sample buffer, was respectively resolved on SDS-PAGE. The PP2A expression levels from each sample were measured on Western blots with an anti-PP2A antibody. Immunoreactive signals of PP2A revealed with ECL were subjected to densitometry scan and quantified with UN-SCAN-IT software. The immunoreactive signals for annexin II from the same samples were used for normalization of the PP2A signals. A significant difference in PP2A protein levels was shown between AC and AD cells (P<0.01, t test). FIG. 3B shows that PP2A activities in AD cells were significantly reduced compared to those in AC cells (*P<0.001).

FIG. 4: Effects of okadiac acid (OA) on BK-stimulated MAP kinase phosphorylation: AC cells were treated with about 10 nM BK for about 5 min and about 10 min in the presence or absence of about 10 nM OA. The resulting Erk1/2 phosphorylation was examined on Western blots. Levels of Erk1/2 phosphorylation were normalized with those of the regular (total amount) Erk1/2. The top panel shows a representative result from Western blots. The bar graph in the lower panel summarizes results from five different AD cells. (**P<0.001). BK, bradykinin; OA, okadiac acid; P-Erk1/2, phospho-Erk1/2.

FIG. 5A-5B: Comparison of the effects of OA and FK506 on BK-increased Erk1/2 phosphorylation in AC and AD cells: AC and AD cells were treated with about 10 nM BK for about 10 min in the presence or absence of about 10 nm OA or about 20 nM FK506. The resulting Erk1/2 phosphorylation under each condition from each cell line was measured as described herein. FIG. 5A shows representative Western blot results on the left panel and a bar graph on the right summarizing results from nine AC and nine AD cell lines. In FIG. 5B, ratios of the BK-stimulated Erk1/2 phosphorylation in the presence of OA or FK605 were calculated against those in the absence of OA or FK506, and compared between AC and AD cells. There is a significant difference in these ratios between AC and AD cells. BK, bradykinin; OA, okadiac acid; P-Erk1/2, phospho-Erk1/2, regular Erk1/2.

FIG. 6A-6B: Immunocytochemical staining: (FIG. 6A) AC and AD cells treated with about 10 nM BK for about 5 min or about 10 min. In a different flask, cells were preincubated with about 10 nM OA for about 15 min prior to an approximately 10-min BK treatment. After termination of the reaction, phosphorylation of Erk1/2 was detected by immunocytochemical staining using an anti-phospho-Erk antibody as described herein. The arrows in the enlarged images point at increased Erk1/2 phosphorylation. (FIG. 6B) AC and AD cells were treated in about 10 nM BK in the presence or absence of about 10 nM OK. Cells were then subjected to double immunofluorescent staining simultaneously with a mouse anti-phospho-Erk1/2 and a rabbit anti-regular Erk1/2 antibody. This was followed by staining with a fluorescein-labeled anti-mouse (green) and a Texas red-labeled (red) anti-rabbit secondary antibody. BK, bradykinin; OA, okadiac acid; P-Erk1/2, phospho-Erk1/2; r-Erk1/2, regular Erk1/2.

### DETAILED DESCRIPTION OF THE INVENTION

The present invention relates to methods of diagnosing Alzheimer's disease in human cells based upon the discovery of specific abnormalities of PP2A expression, function and related biochemical events in Alzheimer's disease fibroblast cells. Sustained Erk1/2 phosphorylation induced by bradykinin was previously found in Alzheimer's disease fibroblasts and is the subject of WO 02/067764, which is herein incorporated by reference in its entirety. Because direct access to neurons in the brains of living human beings is impossible, early diagnosis of Alzheimer's disease is extremely difficult. By testing Alzheimer's disease-specific abnormalities of PP2A and related molecular events, including Erk1/2 phosphorylation and distribution in peripheral cells of Alzheimer's disease patents, the present invention provides highly practical, sensitive, and efficient tests for early diagnosis of Alzheimer's disease. In addition, the Alzheimer's disease-specific differences described herein provide a basis for identifying therapeutic targets for drug development.

The present invention uses the following criteria as the bases for a number of diagnostic tests to assess Alzheimer's disease in human peripheral cells: 1) PP2A expression at the gene level with or without treatment of agents that stimulate phosphorylation of PP2A substrates; 2) PP2A expression at the protein level and PP2A enzymatic activity, with or without treatment of agents that stimulate phosphorylation of PP2A substrates; 3) the effect of agents that inhibit PP2A function on the extent of substrate phosphorylation; and 4) differences in subcellular distribution (or translocation) of phosphorylated Erk1/2, a PP2A substrate, between control cells and Alzheimer's disease cells. Each of the tests described below may be used alone, or in any combination to provide additional specificity.

### Methods of Evaluating Basal PP2A Gene Expression

In one embodiment, the invention relates to a method of diagnosing Alzheimer's disease in an individual by obtaining a cell sample from an individual and detecting the basal level of PP2A gene expression in the cell sample. This embodiment is based upon the discovery by the inventors that fibroblasts from patients of both familial and sporadic Alzheimer's disease present significantly higher basal levels of PP2A gene expression compared to non-Alzheimer's disease cells from age-matched individuals. Therefore, a higher basal level of PP2A indicates the presence of Alzheimer's disease. In one embodiment, mRNA levels encoding PP2A in test cells is quantified and compared to mRNA levels encoding PP2A in control cells.

In the methods of the invention, the cells that are taken from the individual or patient can be any viable cells. Preferably, they are skin fibroblasts, but any other peripheral tissue cell (i.e. outside of the central nervous system) may be used in the tests of this invention if such cells are more convenient to obtain or process. Other suitable cells include, but are not limited to, blood cells such as erythrocytes and lymphocytes, buccal mucosal cells, nerve cells such as olfactory neurons, cerebrospinal fluid, urine and any other peripheral cell type. In addition, the cells used for purposes of comparison do not necessarily have to be from healthy donors.

The cells may be fresh or may be cultured (see, U.S. Pat. No. 6,107,050, which is herein incorporated by reference in its entirety). In a specific embodiment, a punch skin can be used to obtain skin fibroblasts from a subject. These fibroblasts are analyzed directly using the techniques described herein or introduced into cell culture conditions. The resulting cultured fibroblasts are then analyzed as described in the examples and throughout the specification. Other steps may be required to prepare other types of cells which might be used for analysis such as buccal mucosal cells, nerve cells such as olfactory cells, blood cells such as erythrocytes and lymphocytes, etc. For example, blood cells can be easily obtained by drawing blood from peripheral veins. Cells can then be separated by standard procedures (e.g. using a cell sorter, centrifugation, etc.) and later analyzed.

In a preferred embodiment, the level of PP2A gene expression in the cell sample is measured by reverse transcription quantitative polymerase chain reaction (RVQ-PCR) using a Taqman® real-time PCR device with either a 384- or 96-well microplate. A reference gene that is abundantly expressed in the eukaryotic cell such as GAPDH (glyceraldehyde-3-phosphate dehydrogenase) should also be simultaneously amplified and used for normalization. According to the invention, a higher basal level of PP2A gene expression compared to normal cells from age-matched individuals indicates the presence of Alzheimer's disease.

Methods of Evaluating Changes in PP2A Gene Expression Following Stimulation of Cells with Agents that Stimulate Phosphorylation of PP2A Substrates

A further embodiment of the invention relates to a method of diagnosing Alzheimer's disease which involves the steps of obtaining a cell sample from a subject, contacting the sample with an agent that stimulates phosphorylation of a PP2A substrate and comparing the level of PP2A gene expression in the stimulated cells to the level of PP2A gene expression in unstimulated cells of the same type from the individual. In a specific embodiment, the agent is bradykinin. In this embodiment, the absence of bradykinin-induced PP2A gene expression in stimulated cells as compared to the unstimulated cells indicates the presence of Alzheimer's disease. This method is based upon the discovery by the inventors that control cells upregulate PP2A gene expression in response to bradykinin stimulation; whereas, this normal upregulation response is lacking in the cells of Alzheimer's patents. Other possible stimulating agents include, but are not limited to, insulin, phobol esters, lysophosphatidylcholine, lipopolysaccharide, anthracycline dannorubicin and vanadyl sulfate.

Bradykinin is a potent vasoactive nonapeptide that is generated in the course of various inflammatory conditions. Bradykinin binds to and activates specific cell membrane bradykinin receptor(s), thereby triggering a cascade of intracellular events leading to the phosphorylation of proteins known as “mitogen activated protein kinase” (MAPK). Phosphorylation of protein, the addition of a phosphate group to a Ser, Thr, or Tyr residue, is mediated by a large number of enzymes known collectively as protein kinases. Phosphorylation normally modifies the function of, and usually activates, a protein. Homeostasis requires that phosphorylation be a transient process, which is reversed by phosphatase enzymes that dephosphorylate the substrate. Any aberration in phosphorylation or dephosphorylation may disrupt biochemical pathways and cellular functions. Such disruptions may be the basis for certain brain diseases.

In another specific embodiment the bradykinin-induced PP2A gene expression is preferably assessed by calculating the +bradykinin/-bradykinin (BK) ratios. PP2A gene expression from BK-stimulated and non-stimulated cells is performed via real time RT-PCR. For internal normalization, gene expression of a “housekeeper” gene such as GAPDH or S18 rRNA from the same cell samples is simultaneously performed. Concentrations of mRNA for PP2A and the housekeeper gene are automatically calculated by the real-time PCR apparatus according to a standard curve generated for each gene from a serial dilution of cDNA samples. Values representing the concentration of PP2A gene expression are normalized against values representing the concentration of housekeeper gene: NR=GT/GH. Where NR is normalized gene expression; GT is the target gene (PP2A) expression value before normalization; and GH is the gene expression value of a housekeeper gene. Next, ratios of NG from BK+ and BK− cells are calculated by: R=NG_{BK+}/NG_{BK-}. Where R is the

+bradykinin/−bradykinin (BK) ratio; NG_{BK+} is the normalized PP2A gene expression from BK+ cells; and NG_{BK-} is the normalized PP2A gene expression from BK-cells.

### Methods of Evaluating PP2A Protein Levels and Enzymatic Activity

Another embodiment of the invention relates to a method of diagnosing Alzheimer's disease in a subject involving the steps of obtaining a cell sample from the subject and detecting the level of PP2A protein and/or PP2A enzymatic activity in the sample. This embodiment is based upon the discovery by the inventors that both PP2A protein levels and enzymatic activity in Alzheimer's disease cells are significantly reduced compared to non-Alzheimer's disease cells.

In a preferred embodiment, the level of PP2A protein present in cells is detected by Western blotting. Protein levels of PP2A can be measured in fibroblasts using an anti-PP2A antibody (Biosource). Levels of a different protein should also preferably be measured in the same sample as a reference protein for normalization. Examples of possible reference proteins include, but are not limited to, annexin-II or actin. In another embodiment, the level of PP2A activity in AD and AC cells is assayed according to a procedure (Pierce Biotechnology) using p-nitrophenyl phosphate (PNPP) as the substrate. The enzyme activity assays are carried out in a 96-well microplate. The reaction is initiated by adding about 10 μl of each AC or AD cell lysate into about 90 μl of reaction mixture, incubated at about 30° C. for about 15 minutes, and measured in a BioRad microplate reader at a wavelength of 420 nM. After subtraction of values from reactions in which about 10 nM of the PP2A inhibitor okadiac acid is present, the activity of PP2A is calculated according to a standard curve produced by a series of known concentrations of purified PP2A protein.

In one embodiment, ELISA is performed according to the following procedures: 1) Add fibroblast cell lysates after treatment in duplicates or triplicates to a 96-well microplate that is previously coated with an anti-Erk antibody. 2) Incubate samples in microplate wells at room temperature for about 2 hours. 3) Aspirate samples and wash wells with a phosphate buffered saline (PBS)-based washing buffer. 4) Add working dilution of an anti phospho-Erk1/2, or an anti-regular Erk1/2 antibody to each well, and incubate at room temperature for about 1 hour. 5) Aspirate and wash well with washing buffer. 6) Add a working dilution of a secondary antibody conjugated with horseradish peroxidase (HRP) to each well and incubate well at room temperature for about 30 min. 7) Aspirate and wash well with washing buffer. 8) Add stabilized Chromogen such as diaminobenzidine (DAB) and incubate at room temperature for about 30 min. 9) Add stop solution and measure the absorbance at 450 nm. Phosphorylation of Erk1/2 is assessed after normalization: NR=A_{P}/A_{R}. Where NR=the normalized ratio; A_{P} is absorbance values for phospho-Erk1/2; and A_{R} is absorbance for the total (regular) Erk1/2.

Methods of Diagnosing Alzheimer's Disease Using Agents that Inhibit PP2A and Agents that Stimulate Phosphorylation of a PP2A Substrate

In yet another embodiment, the invention relates to a method of diagnosing Alzheimer's disease involving the steps of obtaining a cell sample from a subject and contacting the cells with a first agent that stimulates phosphorylation of a PP2A substrate, in the presence of a second agent that is a PP2A inhibitor, measuring the level of phosphorylation of the PP2A substrate in the sample cells at a predetermined time after initiating the contacting step, and comparing the level of substrate phosphorylation to the level of substrate phosphorylation in known non-Alzheimer's disease cells at the same predetermined time, wherein a lack of response to the PP2A inhibitor in the sample cells compared to the known non-Alzheimer's disease cells indicates the presence of Alzheimer's disease.

This embodiment is based upon the discovery by the inventors that treatment of non-Alzheimer's disease cells with substances such as bradykinin in the presence of a PP2A inhibitor, such as okadiac acid, prolonged Erk1/2 phosphorylation, which is otherwise returned to a basal level after about 10 min after bradykinin stimulation in normal cells. This response is absent in Alzheimer's disease cells. Because the bradykinin-stimulated Erk1/2 phosphorylation is sustained in Alzheimer's disease cells due to inhibition of the normal dephosphorylation mechanism, application of PP2A inhibitors such as okadiac acid has no additional effect on the extent of Erk1/2 phosphorylation. Thus, the ratio of +okadiac acid/−okadiac acid Erk1/2 phosphorylation in non-Alzheimer's disease cells is significantly greater than that in Alzheimer's disease cells.

In a preferred embodiment, a method of diagnosing Alzheimer's disease in a subject is disclosed wherein the method comprises the steps of obtaining a cell sample from a subject; contacting control cells and said cell sample with a first agent that stimulates phosphorylation of a substrate of PP2A (in certain embodiments, the agent is bradykinin and the substrate of PP2A is Erk1/2), wherein the contacting is done in the presence and the absence of a second agent that is an inhibitor of PP2A (in certain embodiments, the second agent is okadiac acid); measuring the level of phosphorylation of the PP2A substrate from said control cells and said cell sample at a predetermined time (in preferred embodiments, after about 5 min. or about 10 min. or about 15 min.) after initiating the contacting step; and comparing the level of phosphorylation of the PP2A substrate from said cell sample in the presence and the absence of said second agent that is an inhibitor of PP2A, wherein a lack of a significant difference between the extent of PP2A substrate phosphorylation in the presence and the absence of said second agent indicates the presence of Alzheimer's disease in the subject from whom the cells were taken. The control cells show a statistically significant difference in the level of phosphorylation of the PP2A substrate in the presence and the absence of said second agent that is an inhibitor of PP2A.

In a preferred embodiment, phosphorylation of Erk1/2 is assayed on Western blots using an anti-phospho-Erk1/2 antibody. Levels of the immunoreactive signals for phosphorylated Erk1/2 are quantified via densitometric scan. The mean density of the phospho-Erk1/2 signals are normalized with the mean density of total Erk1/2 signals that are detected from the same cell lysate samples with an anti-regular Erk1/2 antibody on a separate Western blot. The formula for normalization is: NR=D_{P}/D_{R}. Where NR (normalized ratio) represents Erk1/2 phosphorylation extent; D_{P} is the mean density for phospho-Erk1/2, and D_{R} is the mean density for the total amount of Erk1/2 detected on a Western blot from the same sample. Next, the ratio of NR (test ratio) in the presence and absence of okadiac acid is calculated by the following formula: TR=NR_{OA}+/NR_{OA-}. Where TR is the test ratio, NR_{OA+} is the normalized ratio in the presence of OA, and NR_{OA-} is the normalized ratio in absence of OA.

### Methods of Measuring Distribution of Phosphorylated Erk1/2 in Cells

Many ways to quantify the distribution of phosphorylated Erk1/2 are contemplated and fall within the scope of the invention. Two preferred methods are disclosed as follows. In preferred method 1): Phosphorylation of Erk1/2 after BK stimulation is detected with Immunocytochemistry and signals acquired with fluorescence microscopy. The fluorescence intensity representing phospho-Erk1/2 signals in the nucleus and cytosol are quantified separately with computer software such as Metamorph or NIH Image. The ratio of phospho Erk1/2 in the nucleus over phospho Erk1/2 in the cytosol is calculated by: DR=PN/PC. Where DR is the distribution ratio of phosphorylated Erk1/2; PN is phospho-Erk1/2 in the nucleus; and PC is phospho-Erk1/2 in the cytosol. In preferred method 2): After BK stimulation, cells are subfractionated as the nucleic and cytosolic fractions respectively. Phosphorylation extents of Erk1/2 from these fractions are assayed via Western blotting or ELISA. Ratios of p-Erk1/2 from the nucleus over p-Erk1/2 from the cytosol are calculated by: DR=D_{PN}/D_{PC}. Where DR is the distribution ratio; D_{PN} is the mean densitometric value of phospho-Erk1/2 from the nucleus; and D_{PC} is the mean densitometric value of phospho-Erk1/2 from the cytosol.

In a further embodiment, the present invention provides methods of measuring differences in subcellular distribution (or translocation) of phosphorylated Erk1/2 in non-Alzheimer's disease and Alzheimer's disease cells. This embodiment is based upon the discovery by the inventors that in control cells, phosphorylated Erk1/2 is concentrated in the nucleus, but in Alzheimer's disease cells phosphorylated Erk1/2 is distributed in the extranuclear space (i.e. cytoplasm) of the cells. According to the invention, nuclear translocation of Erk1/2 is tested by stimulating cells with an agent that stimulates phosphorylation of Erk1/2 and the nuclear distribution of activated (i.e. phosphorylated) Erk1/2 is, preferably, examined by either immunocytochemistry, or by a test ratio of phosphorylated Erk1/2 between the nucleus and the cytosol. Nuclear translocation of phosphorylated Erk1/2 can also be examined by Western blotting and ELISA. Any other methods for detecting phosphorylated Erk1/2 are contemplated, including, but not limited to, flow cytometry, protein kinase assays, immunoprecipitation using radiolabeled phosphate, mass spectrometry, fluorescence resonance energy transfer using fluorescently labeled antibodies, immunoprecipitation using antibodies attached to magnetic beads, affinity-based assays using MAP kinase substrates, Northern blots, one or two-dimensional gel chromatography, optionally followed by phosphoprotein staining or detection, enzymatic activity assays.

Immunoassays of the present invention may be immunofluorescent assays, radioimmunoassays, Western blot assays, enzyme immunoassay, immuno-precipitation, chemiluminescent assay, immunohistochemical assay, dot or slot blot assay and the like. (In “Principles and Practice of Immunoassay” (1991) Christopher P. Price and David J. Neoman (eds), Stockton Press, New York, N. Y., Ausubel et al. (eds) (1987) in “Current Protocols in Molecular Biology” John Wiley and Sons, New York, N. Y.). Detection may be by colorometric or radioactive methods or any other conventional methods known to those having skill in the art. Standard techniques known in the art for ELISA are described in Methods in Immunodiagnosis, 2^{nd} Edition, Rose and Bigazzi, eds., John Wiley and Sons, New York 1980 and Campbell et al., Methods of Immunology, W. A. Benjamin, Inc., 1964, both of which are incorporated herein by reference. Such assays may be direct, indirect, competitive, or noncompetitive immunoassays as described in the art (In “Principles and Practice of Immunoassay” (1991) Christopher P. Price and David J. Neoman (eds), Stockton Press, NY, N. Y.; Oellirich, M. 1984. J. Clin. Chem. Clin. Biochem. 22: 895-904 Ausubel, et al. (eds) 1987 in Current Protocols in Molecular Biology, John Wiley and Sons, New York, N. Y.

As stated previously, the cells taken from the patient being diagnosed may be any cell. Examples of cells that may be used include, but are not limited to, fibroblasts, buccal mucosal cells, blood cells, such as erythrocytes, lymphocytes and lymphoblastoid cells, and nerve cells and any other cell expressing the Erk1/2 protein. Necropsy samples and pathology samples may also be used. Tissues comprising these cells may also be used. The cells may be fresh, cultured or frozen. Protein samples isolated from the cells or tissues may be used immediately in the diagnostic assay or frozen for later use. In a preferred embodiment fibroblast cells are used. Fibroblast cells may be obtained by a skin punch biopsy.

Proteins may be isolated from the cells by conventional methods known to one of skill in the art. In a preferred method, cells isolated from a patient are washed and pelleted in phosphate buffered saline (PBS). Pellets are then washed with “homogenization buffer” comprising 50 nM NaF, 1 mM EDTA, 1 mM EGTA, 20 μg/ml leupeptin, 50 μg/ml pepstatin, 10 mM TRIS-HCl, pH=7.4, and pelleted by centrifugation. The supernatant is discarded, and “homogenization buffer” is added to the pellet followed by sonication of the pellet. The protein extract may be used fresh or stored at −80° C. for later analysis.

In this methods of the invention, the antibodies used in the disclosed immunoassays may be monoclonal or polyclonal in origin. The phosphorylated and non-phosphorylated Erk1/2 protein or portions thereof used to generate the antibodies may be from natural or recombinant sources or generated by chemical synthesis. Natural Erk1/2 proteins can be isolated from biological samples by conventional methods. Examples of biological samples that may be used to isolate the Erk1/2 protein include, but are not limited to, skin cells, such as, fibroblasts, fibroblast cell lines, such as Alzheimer's disease fibroblast cell lines and control fibroblast cell lines which are commercially available through Coriell Cell Repositories, (Camden, N. J.) and listed in the National Institute of Aging 1991 Catalog of Cell Lines, National Institute of General Medical Sciences 1992/1993 Catalog of Cell Lines [(NIH Publication 92-2011 (1992)].

It is further contemplated that this invention relates to kits which can be utilized in performing any of the diagnostic tests described above. As stated previously, the kits may contain a single diagnostic test or any combination of the tests described herein. Such kits may comprise antibodies which recognize the PP2A or phosphorylated PP2A substrates, as well as any compounds that stimulate phosphorylation of PP2A substrates (such as, for example, bradykinin) and/or inhibitors of PP2A function (such as, for example, okadiac acid). Antibodies may be polyclonal or monoclonal. The kits may also contain instructions relating to the use of the antibodies or other constituents in the diagnostic tests. The kits may also contain other reagents for carrying out the diagnostic tests such as oligonucleotide primers for PCR or RT-PCR which are specific for the gene encoding PP2A and the gene encoding “housekeeper genes” such as GAPDH, for example. The kits may also include buffers, secondary antibodies, control cells, and the like.

### Methods of Screening to Identify Therapeutic Substances

In another embodiment, the diagnostic tests described herein can also be used to screen and identify substances useful for the treatment or prevention of Alzheimer's disease. According to this embodiment, substances which reverse or improve the Alzheimer's disease-associated differences described herein (i.e. back to levels found in normal cells) would be identified and selected as substances which are potentially useful for the treatment of Alzheimer's disease.

By way of example, one such method of screening therapeutic substances would involve the steps of contacting sample cells from an Alzheimer's disease patient with a substance being screened, and detecting the level of PP2A gene expression in the sample, wherein a reduction in the abnormally elevated level of PP2A gene expression associated with Alzheimer's disease cells indicates that the substance is potentially useful for the treatment or prevention of Alzheimer's disease. The elevation of PP2A gene expression in AD cells is a cellular compensation for the reduced PP2A protein levels and impaired PP2A activity. A substance that increases the PP2A protein level or enhances PP2A activity will reduce prolonged Erk1/2 phosphorylation and thus is potentially useful for treatment of AD. If PP2A protein and activity are increased, the elevated PP2A gene expression may return to a normal level.

In another preferred embodiment of the compound screening method disclosed herein, the Alzheimer's disease-associated abnormality is the lack of increased PP2A expression in cells contacted with an agent that stimulates phosphorylation of Erk1/2. In this embodiment, compounds that restore increased PP2A expression in cells contacted with an agent such as bradykinin, which stimulates Phosphorylation of Erk1/2, would potentially identify a compound useful for the treatment or prevention of Alzheimer's disease.

In another preferred embodiment of the compound screening method disclosed herein, the Alzheimer's disease-associated abnormality is reduced PP2A protein or PP2A enzymatic activity compared to non-Alzheimer's control cells. In this embodiment, compounds that restore normal levels of PP2A protein or PP2A enzymatic activity in cells isolated from subjects having Alzheimer's disease, would potentially identify a compound useful for the treatment or prevention of Alzheimer's disease.

In another preferred embodiment of the compound screening method disclosed herein, the Alzheimer's disease-associated abnormality is the lack of a normal response when test cells are treated with bradykinin in the presence of okadiac acid. In this embodiment, compounds that restore a normal response in cells isolated from subjects having Alzheimer's disease, would potentially identify a compound useful for the treatment or prevention of Alzheimer's disease.

In a further preferred embodiment of the compound screening method disclosed herein, the Alzheimer's disease-associated abnormality is distribution of phosphorylated Erk1/2 in the extranuclear area. In this embodiment, compounds that restore a normal distribution of phosphorylated Erk1/2 in the nucleus of cells isolated from subjects having Alzheimer's disease, would potentially identify a compound useful for the treatment or prevention of Alzheimer's disease.

Those skilled in the art will readily recognize that any of the Alzheimer's disease-associated differences described in this invention can be adapted to form the basis of screening methods or assays for the identification of therapeutic substances for the treatment of prevention of Alzheimer's disease. In addition, such methods would utilize any of the techniques or materials well known in the art and/or already disclosed herein and in the Examples.

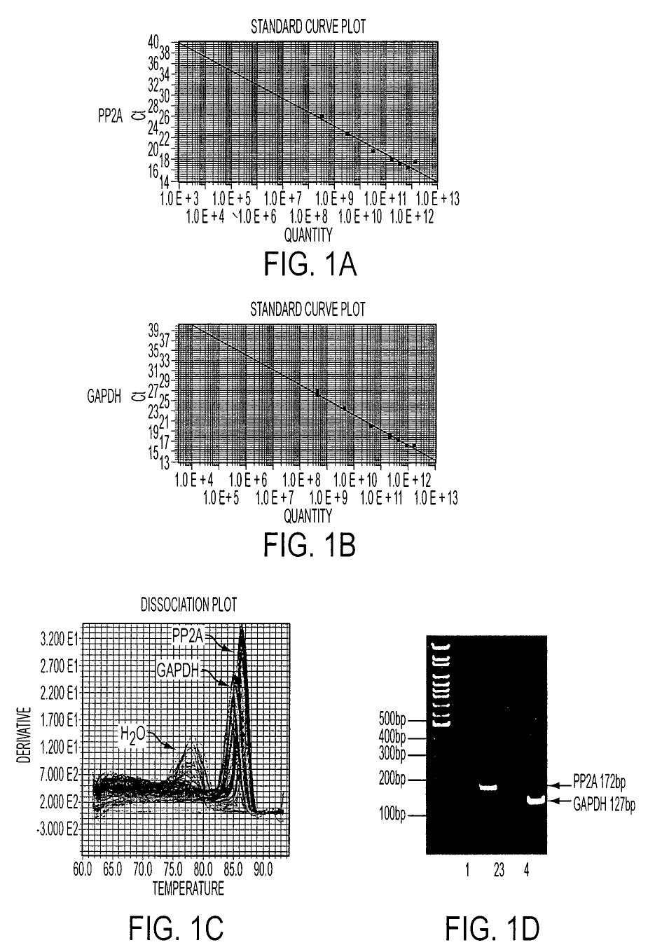

The inventors have found that the serine/threonine phosphatase 2A is impaired in fibroblast cells from AD patients. This impairment includes abnormal expression of PP2A at gene and protein levels and impairment in its phosphatase activity. PP2A gene expression in AD and AC cells is measured with RTQ-PCR, a highly sensitive method for comparing mRNA levels (Heid et al., 1996; Winer et al., 1999; Livak and Schmittgen, 2001). In order to minimize sample-to-sample variation due to possible differences in the starting amount of cDNA copies, levels of GAPDH mRNA are used for normalization of PP2A gene expression. Since no genomic DNA is contained in the reverse-transcribed samples, all amplified cDNA copies should be attributed to mRNA prepared from AD and AC cells. The PCR products are specific for PP2A and GAPDH, as demonstrated by their characteristic melting curves (TM), as well as by a single PCR product of PP2A or GAPDH resolved on the TBE gel with expected sequence size (FIG. 1).

A significantly high basal level of PP2A gene expression exists in AD cells. Higher basal PP2A mRNA levels, however, do not necessarily result in higher protein expression, nor does it necessarily indicate normal PP2A function. Indeed, in AD cells the amount of PP2A is significantly lower compared to control cells, as are PP2A enzymatic activities. Because AD is an etiologically heterogeneous disorder, multiple factors may be involved in the upstream molecular mechanism underlying abnormal expression and activity of PP2A in AD cells. The reduced protein levels of PP2A in AD cells may be a result of impaired post-transcriptional processes in protein synthesis, and/or from compromised PP2A protein stability due to abnormally increased proteolysis or incorrect protein folding, which could facilitate degradation of PP2A proteins. Reduction of PP2A protein in AD cells will cause impaired PP2A activity. Additionally, altered enzyme properties such as substrate-binding affinity of the regulatory domain and/or the activity of the catalytic subunit are also factors that impair PP2A function.

Other abnormalities in upstream molecular events, for example, perturbation of calcium homeostasis in AD, are known. Two CA²⁺-binding EF-hand motifs have been identified in the B/PR72 regulatory subunit of PP2A, which is involved in regulation of PP2A activity (Janssens et al., 2003). In an in vitro system, these authors showed that low CA²⁺ concentrations increased PP2A activity, but high CA²⁺ concentrations inhibited it (Janssens et al., 2003). Abnormally enhanced intracellular calcium signaling has been found in different types of cells from AD, including those caused by presenilin-1 mutations (Sheehan et al., 1997; Etcheberrigaray et al., 1998; Putney, 2000; Yoo et al., 2000; Mattson et al., 2001). Increased intracellular CA²⁺ levels together with oxidative stress may be key factors contributing to PP2A function deficits, and enhanced activities from upstream protein kinases such as MEK, PKC, and PP60-src leading to increases and prolongation of MAP kinase activity. Abnormally increased pp60-src activity, for example, could not only promote MAP kinase phosphorylation (Zhao et al., 2002), but also suppress PP2A activity (McMahon et al., 2001), both contributing to dysregulation of the Map kinase pathway in AD cells.

PP2A mRNA expression is reduced in postmortem AD brains (Gong et al., 1995; Vogelsberg-Regaglia et al., 2001). It is possible that the increased basal levels of PP2A mRNA disclosed herein reflect a cellular compensatory mechanism for its deficient protein expression and enzymatic functions in AD cells. This compensatory phenomenon is found in living AD cells as shown herein, but it may be completely diminished in terminal states of AD so that lower PP2A mRNA levels might be detected in postmortem AD brains.

BK is a potent inflammation mediator that stimulates a series of intracellular CA²⁺-dependent signal transduction processes, including protein phosphorylation, and activation of transcriptional factors leading to gene expression (Connolly, 1998; Liebmann, 2001). As part of a normal feedback mechanism, phosphatase may be activated as a result of protein phosphorylation in response to cellular stimuli, and gene expression of specific phosphatases may be upregulated in order to supply sufficient enzyme to the cell. The present inventors have demonstrated that when AC cells are stimulated with BK for about 10 min, a significant elevation of PP2A gene expression is detected, which demonstrates a normal cellular response to a pharmacological stimulus. This response, however, is not shown in AD cells as PP2A mRNA levels do not change after BK stimulation. This loss of regulation capability of PP2A gene expression in response to stimulation underlies the impairment of PP2A function during AD pathogenesis.

BK causes an increase in Erk1/2 phosphorylation. In AC cells, this increased Erk phosphorylation lasts for a few minutes and returns to the control level by about 10 min poststimulation. In AD cells, however, it is significantly sustained (Zhao et al., 2002). Dysfunction of PP2A contributes to AD-associated enhancement of Erk1/2 phosphorylation. The present inventors determined the effects of the PP2A inhibitors, including OA and the PP2B inhibitor FK506 on Erk1/2 phosphorylation after bradykinin stimulation. Inhibition of PP2A by OA increases Erk1/2 phosphorylation. This increase in AC cells is markedly greater than that in AD cells by a significantly higher ratio of +OA/−OA in AC cells. Because OA is used in a dose (about 10 nM) selectively inhibiting PP2A (Nagao et al., 1995; Sheppeck et al., 1997; Fernandez et al., 2002), along with the result in which FK506 does not inhibit Erk1/2 phosphorylation, PP2A, and no other phosphatases such as PP1 or PP2B, is responsible for inactivation of the BK-induced Erk1/2 phosphorylation in fibroblasts. Therefore, sustained Erk1/2 phosphorylation induced by BK stimulation in AD cells is attributed to the impairment of PP2A function.

When cells are treated with FK506, the prolongation of Erk1/2 phosphorylation in AD cells induced by BK was abolished. In addition to PP2B, FK506 also primarily targets FK-binding proteins (FKBP) that represent a class of peptidyl prolyl cis/trans isomerases (PPIase). Previous studies have reported that FK506 promotes expression and activity of MAP kinase phosphatase 1 leading to decline of Erk phosphorylation and downstream signaling (Winter et al., 1998; Zawadzka and Kaminska, 2003). By inhibiting PPIase activity, FK506 and other FKBP ligands have been reported to have a neuroprotective function (Gold, 1999, 2000; Christner et al., 2001; Klettner et al., 2001). Erk1/2 is a key player among signaling pathways regulating a variety of cellular events. Activation of Erk in response to mitogenic stimuli has been reported to cause translocation of the kinase from the cytosol to the nucleus (Chen et al., 1992; Gonzales et al., 1993; Lenormand et al., 1993; Brunet et al., 1999; Ferrell, 1998; Lewis et al., 1998), where it participates in regulation of gene transcription processes (Treisman, 1996). The nucleus is also a critical site for inactivation of Erk1/2 via nuclear sequestration of Erk1/2 away from its upstream activating kinase MEK, its cytoplasmic activator, and its dephosphorylation by specific nuclear phosphatase (Volmat et al., 2001). The nuclear import of Erk is mediated via several mechanisms including passive diffusion of the Erk monomer, active transport of the Erk dimer, and by direct interaction of Erk with the nuclear pore complex (Khokhlatchev et al., 1998; Adachi et al., 1999; Matsubayashi et al., 2001). The present inventors disclose herein immunocytochemical staining results showing that activated Erk1/2 is concentrated in the nucleus of AC cells, while a substantial amount of phospho-Erk1/2 remains in extranuclear areas of AD cells. The present invention is also directed to differential subcellular distributions of the phosphorylated Erk1/2 showing that mechanisms underlying nuclear import of activated Erk1/2 are impaired in AD cells.

The present invention exploits the observation made by the inventors that impairment of PP2A functions, including its gene expression and protein production as well as its enzymatic activity, are present in fibroblast cells from AD patients. This impairment of PP2A is responsible for the BK-induced prolongation of Erk1/2 phosphorylation in AD cells. Dysfunctions of PP2A occur also in neurons of the AD brain, causing its incapability to efficiently reverse the hyperphosphorylation of tau protein leading to NFT lesions. Impaired PP2A in the brain also causes a delayed Erk inactivation, which further contributes to greater tau phosphorylation. Dysfunctions of other phosphatase including the dual tyrosine phosphatase, another major phosphatase responsible for inactivating Erk, may also contribute to the AD-associated dysfunction of Erk signaling.

All of the references, patents and printed publications mentioned in the present application are hereby incorporated by reference in their entirety into this application.

The following Examples serve to illustrate further the present invention and are not to be construed as limiting the scope of the invention in any way.

### EXAMPLES

### Example 1

### Changes in PP2A mRNA Levels in AD Cells

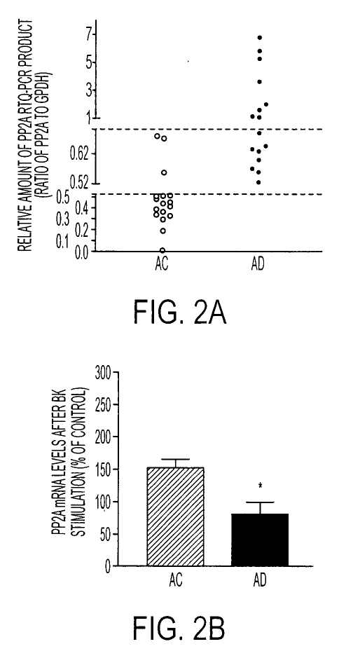

PP2A gene expression was quantified using RTQ-PCR, with GAPDH as a reference gene for normalization. As shown in FIGS. 1A and 1B, with real-time PCR, PP2A and GAPDH primers, respectively, produced a linear standard curve of the amplified sequence with a series of dilutions of the human fibroblast cDNA template run in duplicates. Specific melting temperatures (MT) were plotted by distinct dissociation curves (FIG. 2C) for PP2A, GAPDH, and water, demonstrating a high specificity of each PCR product. This specificity was confirmed by the result shown in FIG. 1D, in which the final PCR products for PP2A and GAPDH were run on a 10% TBE gel. A single band with the expected sequence size was revealed for each gene (lane 2 and 4), but it was not detected in the sample without adding reverse transcriptase during in vitro reverse transcription (lane 1 and 3). This indicates that the amplified PCR products for PP2A and GAPDH were not from the genomic DNA. To measure PP2A gene expression, duplicates of fibroblast cDNA template from each of 19 AD and 17 AC cell lines were applied to PCR. The expression levels of PP2A and GAPDH from each cell line were automatically calculated against their respective standard curve run simultaneously with AC and AD samples. Levels of PP2A gene expression were normalized with those of GAPDH for each cell line, and the resulting ratios were compared using at test. As shown in FIG. 2A, the basal levels of PP2A mRNA were statistically higher in AD cells compared to AC cells (P<0.01, t test). Treatment of the fibroblasts with 10 nM bradykinin (BK) for about 10 min markedly increases PP2A mRNA levels in AC cells. This BK-stimulated PP2A gene upregulation, however, is absent in AD cells (FIG. 2B). At test analysis showed a significant group difference (P=0.016). These results indicate that, despite higher basal levels of PP2A mRNA, the dynamic gene expression of PP2A in response to BK stimulation, is impaired in AD cells.

### Example 2

### Changes in PP2A Protein Levels and Enzymatic Activities in AD Cells

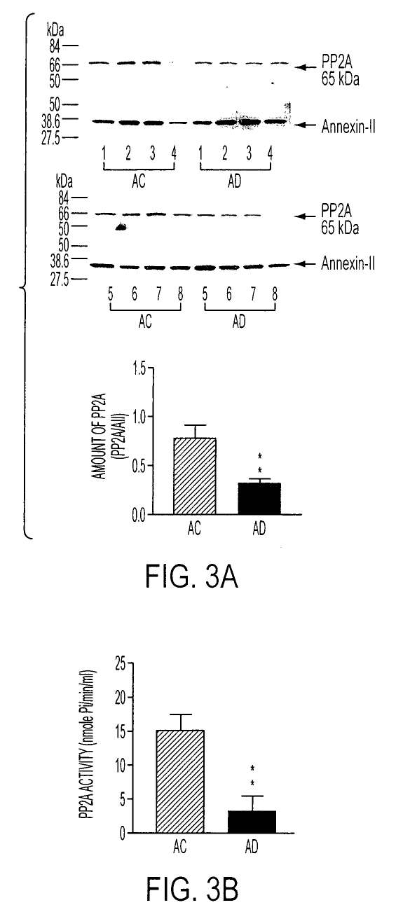

To determine whether changes in PP2A gene expression in AD cells were reflected in its protein expression and function, both PP2A protein levels and activity were compared between AC and AD cells. The amount of PP2A protein measured with Western blotting was significantly reduced in all AD cells compared to that in AC cells (P<0.01). This reduction of PP2A was not due to a lower amount of protein from AD cells that was loaded on SDS-gel, because levels of a reference protein annexin II from the same samples were not significantly different from those in AC cells (FIG. 3A). A consistent result of reduction of PP2A in AD cells was also produced when the PP2A-immunoreactive signals were normalized against the total protein loaded on the SDS-gel. In addition, PP2A activity was also markedly decreased in AD cells compared to AC cells (P<0.001) (FIG. 3B).

### Example 3

### PP2A is Involved in Dephosphorylation of Erk1/2 after BK Stimulation

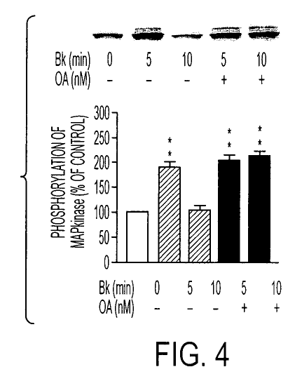

To test whether PP2A is involved in dephosphorylation of Erk1/2, AC cells from five different individuals were treated with a PP2A inhibitor, okadiac acid at a concentration only inhibiting PP2A (Nagao et al., 1995; Sheppeck et al., 1997; Fernandez et al., 2002). The Erk1/2 phosphorylation was determined on Western blots using specific antibodies for phospho- and regular Erk1/2. Erk1/2 phosphorylation was increased at about 5 min after BK stimulation, but it returned to the control level by about 10 min (FIG. 4) possibly due to a normal dephosphorylation mechanism in the cell. In the presence of about 10 nM OA, however, this Erk1/2 dephosphorylation was significantly inhibited (FIG. 4). A one-way ANOVA revealed significant treatment effects (P<0.001). These results indicate that PP2A is responsible for dephosphorylation of Erk1/2 after its BK-stimulated phosphorylation.

### Example 4

### Impaired PP2A Function Contributes to the Prolonged Erk1/2 Phosphorylation in AD Cells

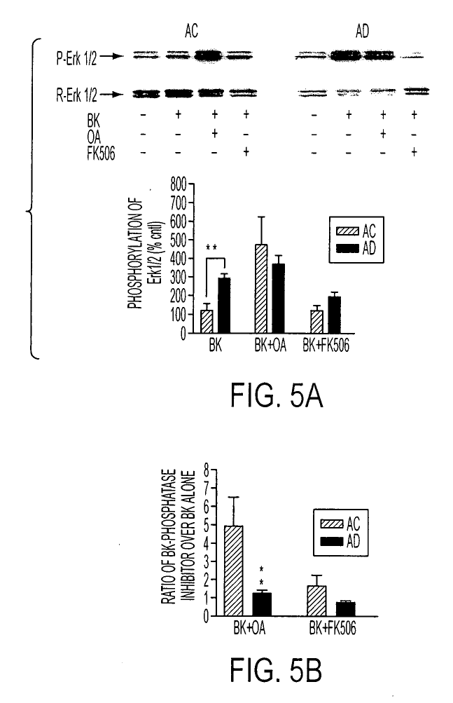

To test whether impairment of PP2A contributes to the prolongation of Erk1/2 phosphorylation after BK stimulation, we treated both AC and AD cells with BK for about 10 min in the presence or absence of about 10 nM OA. The resulting Erk1/2 phosphorylation was examined as described above. Results from 9 AD and AC cell lines clearly showed (FIG. 5A) that OA inhibited Erk1/2 dephosphorylation in AC cells at about 10 min after bradykinin stimulation. In AD cells, a prolonged Erk1/2 phosphorylation was seen at about 10 min after bradykinin stimulation. Addition of OA did not further increase Erk1/2 phosphorylation in these cells. There was a significant difference in ratios of +OA/−OA between AC and AD cells. These results indicate that the prolongation of Erk1/2 phosphorylation in AD cells induced by BK stimulation is due to PP2A function.

On the other hand, the presence of a PP2B inhibitor, FK506 did not cause a significant increase in the BK-induced Erk1/2 phosphorylation in AC cells (FIG. 5A). It was also noted that the BK-induced Erk1/2 phosphorylation prolongation in AD cells was abolished in the presence of FK506 (FIG. 5A).

### Example 5

### Immunocytochemistry

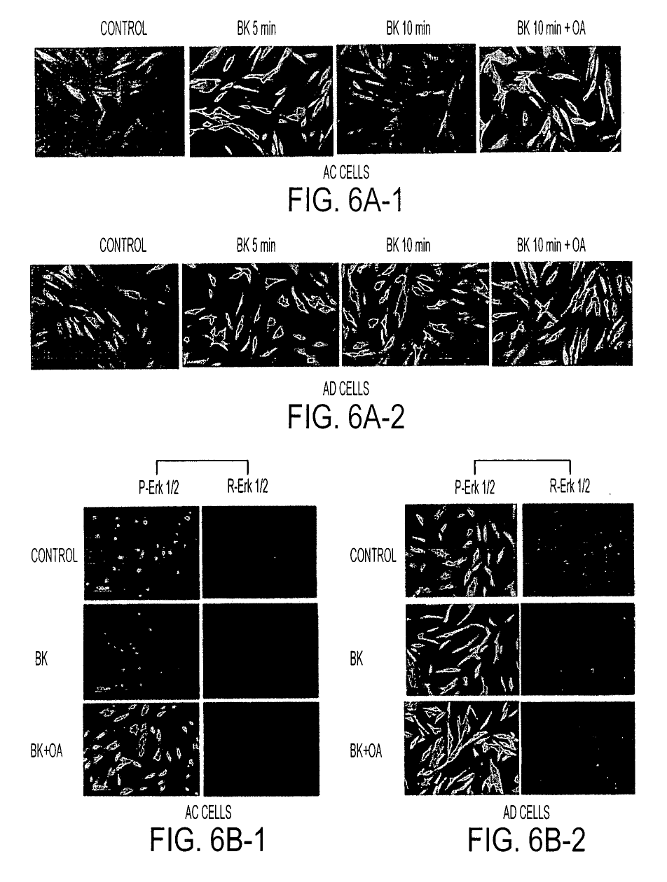

Immunoreactive signals for phospho-Erk1/2 under different treatments are shown in FIG. 6. FIG. 6A shows the time course of the BK-induced Erk1/2 phosphorylation between AC and AD cells in the presence or absence of OA, which was consistent with the Western blotting results (see FIG. 4). FIG. 6B shows the Erk1/2 phosphorylation in comparison with the regular Erk1/2 signals within the same AC or AD cells, which is again consistent with those from Western blots shown in FIG. 5. However, it was noted that the basal phosphorylation levels of Erk1/2 observed with immunohistostaining were higher in AD cells than in AC cells, unlike the results from Western blots, in which there were no clear differences in the basal level of Erk1/2 phosphorylation between AD and AC cells. More significantly, it was also noted that there was a difference in subcellular distribution of phosphorylated Erk1/2 between AD and AC cells. In AC cells the phosphorylated Erk1/2 was predominantly concentrated in the nucleus of the cell, whereas in AD cells phosphorylated Erk1/2 was more diffusely distributed in the paranucleic and cytosolic area. This was particularly true when Erk1/2 was activated by BK (see FIG. 6A BK, about 5 min). These results indicate that translocation of activated Erk to the nucleus is inhibited in AD cells, which may underlie the AD-associated dysfunction of MAP kinase in regulation of gene transcription, as well as the delayed Erk dephosphorylation after BK stimulation.

### Example 6

### Testing Human Skin Fibroblasts

Human skin fibroblasts may be used as the material for the diagnostic tests for Alzheimer's disease of the present invention. This type of cell can be collected from test subjects and age-matched non-Alzheimer's control subjects, processed, cultured and passaged according to established methods. Cells may be cultured either in a small flask (usually 25 cm), or a small dish (35 mm) in DMEM medium containing 10% fetal bovine serum until they reach 80-90 confluency. Cells may then be “starved” by being cultured in a serum-free medium overnight prior to treatment of the cell.

Basal levels of PP2A gene expression are measured by quantitative real time PCR. This includes the following procedures: 1) Preparation of total RNA from fibroblasts or other methods such as a filtration-based methods to prepare total RNA. 2) Removal of genomic DNA by treating the total RNA sample with, for example, DNase-I. 3) Synthesis of single-strand cDNA from the total RNA in an in vitro reverse transcription reaction. 4) Performance of real-time PCR. A reference gene such as GAPDH is simultaneously amplified with the PP2A gene in the same PCR run for normalization of PP2A gene expression.

Bradykinin-induced PP2A gene expression is measured by the following procedures: Serum-starved fibroblasts are treated with an appropriate concentration of bradykinin (BK) at 37° C. for about 10 min. The reaction is terminated by removing the culture medium, rinsing cells with pre-cooled PBS pH 7.5, and freezing cells on a dry ice/ethanol surface. The same cells cultured in a separate flask are added with the same volume of PBS instead of BK solution, and used as the control. Preparation of total RNA, DNase-I treatment, in vitro reverse transcription, and real-time PCR are conducted as described above. The BK-induced PP2A gene expression is assessed by calculating the +BK/−BK ratios.

Protein levels of PP2A in fibroblasts are measured with Western blotting using an anti-PP2A antibody. Levels of a different protein such as annexin-II or actin may also be measured in the same sample and used as a reference protein for normalization.

Okadiac acid (OA)-inhibited Erk1/2 dephosphorylation after BK stimulation is examined in the following procedures: Cells from the same cell line are cultured in two separate flasks or dishes up to 80-90% confluency. After serum-starving overnight, cells are treated as such: 1) BK treatment for about 10 min, 2) pretreatment with about 10 nM OA for about 15 min followed by BK treatment and another dose of OA for about 10 min. Reactions are terminated, cells are lysed in a lysis buffer, and the extent of Erk1/2 phosphorylation and levels of the total Erk1/2 are determined using Western blots. After normalizing with signals of the total Erk1/2, the ratio of BK-stimulated Erk1/2 phosphorylation in the presence and absence of OA is calculated.

Basal phosphorylation levels of Erk1/2 are examined with fluorescent immunocytochemical staining. Fibroblasts are cultured on small round coverslips. After reaching about 70-80% confluency and serum-starving overnight, the culture medium is removed. Cells are rinsed rapidly with pre-cooled PBS pH 7.5 and fixed with about 4% formaldehyde. The fixed cells are washed for three times of about 5 min each, and incubated with anti-phospho-Erk1/2 antibody. This is followed by staining cells with a second antibody labeled with fluorescence. The immunoreactive signals are acquired with fluorescent microscopy, and levels of signals for phospho-Erk1/2 are measured with the Metaphore software.

Nuclear translocation of phospho-Erk1/2 is examined with immunocytochemical staining, Western blotting, and ELISA. 1) Cells are cultured on small coverslips to a confluency of 70-80%. Cells are serum-starved overnight and treated with appropriate concentrations of BK in the presence and absence of about 10 nM OA. After termination of the reaction and fixation of cells as described above, cells are immunostained with an anti-phospho-Erk1/2 followed by staining with a fluorescent-labeled secondary antibody. Increases and nuclear importation of phospho-Erk1/2 are observed and recorded with fluorescent microscopy connected to a computer. 2) Cells from an identical cell line are cultured in several separate flasks or dishes for the following treatment conditions: control, BK treatment, and BK+OA treatment. After termination of reactions, the cytosolic and nuclear fractions are separated with a commercial nuclear fractions preparation kit. The nuclear translocation of phospho-Erk1/2 is examined by detecting Erk1/2 phosphorylation levels in the cytosolic and nuclear fractions respectively. The ratio of the nuclear phospho-Erk1/2 to the cytosolic phospho-Erk1/2 is calculated and compared among different treatment conditions. Alternatively, the same results can be obtained with ELISA.

### Example 7

### Cultures and Treatments of Fibroblast Cells

Banked skin fibroblast cells from Alzheimer's disease patients and age-matched controls (AC) were purchased from Coriell Institute for Medical Research. Cells from 19 AD patients aged from 59 to 81 years old were used in this study, with 11 cell lines from familial AD (FAD) and 9 cell lines from sporadic AD (SAD) individuals. All patients showed severe dementia, progressive memory loss, and other impaired cognitive functions. Abnormal eletroencephalogram and various degrees of cerebral atrophy were also found in these AD patients. Control fibroblast cells were from 17 normal individuals with close age and sex matches. Upon arriving at the laboratory, cells were cultured in DMEM medium containing 10% fetal bovine serum and passaged as previously described (Zhao et al., 2002). Cells with passage number not greater than 17 were used in this study.

### Example 8

### Pharmacological Treatments

To stimulate MAP kinase phosphorylation, fibroblasts were cultured to approximately 90% confluence and treated with bradykinin (BK, 10 nM), a potent inflammation mediator, for about 5 min. or about 10 min. To test a possible involvement of PP2A or PP2B in regulation of MAP kinase phosphorylation, cells were pretreated with either okadiac acid (OA, about 10 nM) or FK506 (about 20 nM) for about 15 min followed by treatment with BK (about 10 nM) alone with another dose of okadiac acid or FK506 for about 10 min. A flask of cells for each cell line was treated with DMSO vehicle and used as controls. The treatment was terminated by removing the culture medium from the flask, rapidly rinsing cells with precooled 1×PBS, pH 7.5, and placing the flask or dry ice/ethanol. Depending on the purpose of the experiment, either 1 ml RNA isolator or 1 ml cell lysis buffer containing 1% protease inhibitor cocktail (Sigma) was added to each flask for subsequent RNA preparation, or enzymatic and immunoblotting assays.

### Example 9

### Preparation of Total RNA and Synthesis of the First-Strand cDNA

Total RNA was extracted from each AD and AC cell line using an RNA isolator (Sigma Genosys) according to the manufacturer's instructions and then treated with DNase-I at 37° C. for 30 min to remove possible genomic DNA contamination. Total RNA (1.5 μg) was then reverse-transcribed to the single-strand cDNA using a first-strand cDNA synthesis kit with oligo(dT) primers.

### Example 10

### Real-Time PCR

The mRNA levels were quantified by a real-time polymerase chain reaction using an ABI 7900 platform (Applied Biosystems) after an in vitro reverse transcription (RTQ-PCR) as described above. The target segment of PP2A was amplified with a primer pair of forward, 5′-GTTGGGAGGTGGCAGTGAG-3′ SEQ ID NO:1 and reverse, 5′-AAACACTGGCCTCTGGTGTC-3′ SEQ ID NO:2, PCR was performed with a 20-μl mixture containing 10 μl the SYBR green-I MaterMix (Applied Biosystems), 10 pmol of each forward and reverse primers, and 1 μg reverse-transcribed cDNA template. To correct errors due to variability of cDNA concentration across samples, a segment of a reference gene, glyceraldehyde 3-phosphate dehydrogenase (GAPDH), was amplified simultaneously in the same PCR run with a primer pair of forward, 5′CAACTTTGGTATCGTGGAAGGACTC-3′ SEQ ID NO:3 and reverse, 5′AGGGATGATGTTCTGGAGAGCC-3′ SEQ ID NO:4. Real-time amplifications of PP2A and GADHP were automatically calculated by the PCR machine, according to a standard curve during the same PCR run for each gene generated with a series dilution of cDNA templates ranging from 10⁵ to 10¹² copies. At the end of PCR, PP2A mRNA levels were normalized with GAPDH mRNA levels. The resulting ratios (PP2A/GADPH) were used as a measure of PP2A gene expression levels in each individual cell line. Specificities of PP2A and GRAPH PCR products were indicated by their melting temperatures (MT), and verified by resolving the final PCR product on a 10% TUBE gel.

### Example 11

### Phosphatase Activity Assays

PP2A activities in AD and AC cells were assayed according to a procedure (Pierce Biotechnology) using p-nitrophenyl phosphate (PNPP, 14.4 mM) as the substrate. The enzyme activity assays were carried out in a 96-well microplate. The reaction was initiated by adding 10 μl of each AC or AD cell lysates into 90 μl of reaction mixture, incubated at 30° C. for 15 min, and measured in a BioRad microplate reader under 420 nm wavelength. After subtraction of values from reactions in which 10 nM PP2A inhibitor okadiac acid was present, activities of PP2A were calculated according to a standard curve produced by a series of known concentrations of purified PP2A protein.

### Example 12

### Determination of Levels of PP2A Protein

To assess levels of PP2A in fibroblasts, the total protein concentrations in cell lysates were determined using BCA protein assay reagent (Pierce Biotechnology). Similar amounts of total protein from each AC and AD cell line were solved on 4-20% SDS-PAGE. PP2A protein was detected with Western blots using an anti-PP2A polyclonal antibody (Biosource International). Annexin II, a phospho-lipid-binding protein that is abundantly expressed in fibroblasts, was also measured with an anti-annexin II antibody (Santa Cruz Biotechnology) on the same blot and its immunoreactive signal was used as a reference for normalization of protein loading variations.

### Example 13

### Measurement of Erk1/2 Phosphorylation

Erk1/2 phosphorylation from different treatments was determined on Western blots using an anti-phospho-Erk1/2 antibody (Cell Signaling Technology), the total amount of Erk1/2 protein loaded on the SDS gel was determined by an anti-regular Erk1/2 antibody (Upstate Biotechnology), and was used to normalize the detected phospho-Erk1/2 signals.

### Example 14

### Immunohistochemistry Staining

Fibroblast cells were grown on the surface of 2.5-cm-diameter glass coverslips coated with 0.02 mg polylysine. After treatment with bradykinin in the presence or absence of OA, cells were rapidly fixed with 4% formaldehyde for 15 min and then penetrated with 0.1% Triton X-100 for 30 min. After 30-min incubation with 10% normal horse serum, cells were treated with anti-phospho-Erk1/2 antibody at 4° C. overnight. Cells were washed and treated with an anti-rabbit IgG antibody labeled with flourscein (green) for 60 min. Following washing and sealing with Vectashield mounting medium (Vector Laboratories), the phospho-Erk1/2 immunostaining signals were observed using a Nikon fluorescent microscope. In other cases, double immunostaining was performed to observe the phosphor- and regular Erk1/2 on the same slice by incubation of cells simultaneously with a mouse anti-phospho- and a rabbit anti-regular Erk1/2 antibody. This was followed by incubation with secondary antibodies of anti-mouse and anti-rabbit IgGs labeled with fluorescein (green) and Texas red (red). Immunoreactive signals were acquired as described above.

### Example 15

### Data Analysis

(1) Quantitative PCR values for PP2A mRNA in each sample of 19 AC and 19 AD cell lines were normalized by those of GPDH in the same sample. (2) For PP2A protein expression, immunoreactive signals were subjected to densitometric scan. The densitometric values for PP2A were normalized by those of annexin 11 and quantified with UN-SCAN-IT software (Silk Scientific, Inc.) (3) To assess Erk1/2 phosphorylation, ratios for phospho-Erk1/2 over those of the total Erk1/2 were calculated. All above ratios and data from phosphatase 2A activity assays were then statistically compared between AD and AC cells using either at test or one-way ANOVA.

### REFERENCES

Adachi, M., Fukuda, M., Nishida, E., 1999. Two co-existing mechanisms for nuclear import of MAP kinase: passive diffusion of a monomer and active transport of a dimmer. EMBO J. 18, 5347-5358.
Alessi, D. R., Gomez, N., Moorhead, G., Lewis, T., Keyse, S. M., Cohen, P., 1995.

Inactivation of p42 MAP kinase by protein phosphatase 2A and a protein tyrosine phosphatase, but not CL100, in various cell lines. Curr. Biol. 5, 283-295.
Billingsley, M. L., Kincaid, R. L., 1997. Regulated phosphorylation and dephosphorylation of tau protein: effects on microtubule interaction, intracellular trafficking and neurodegeneration. Biochem. J. 323, 557-591.
Braconi Quintaje, S. B., Church, D. J., Rebsamen, M., Valloton, M. B., Hemmings, B. A., Lang, U., 1996. Role of protein phosphatase 2a in the regulation of mitogen-activated protein kinase activity in ventricular cardiomyocytes. Biochem. Biophys. Res. Commun. 221, 539-547.
Brunet, A., Roux, D., Lenormand, P., Dowd, S., Keyse, S., Pouyssegur, J., 1999. Nuclear translocation of p42/p44 mitogen-activated protein kinase is required for growth factor-induced gene expression and cell cycle entry. EMBO J. 18, 664-674.
Connolly, G. P., 1998. Fibroblast models of neurological disorders: fluorescence measurement studies. Trends Pharmacol. Sci. 19, 171-177.
Chen, R. H., Sarnecki, C., Blenis, J., 1992. Nuclear localization and regulation of Erk- and rsk encoded protein kinases. Mol. Cell. Biol. 12, 915-927.
Christner, C., Herdegen, T., Fischer, G., 2001. FKBP ligands as novel therapeutics for neurological disorders. Mini-Rev. Med. Chem. 1, 337-397.
Chung, H., Brautigan, D. L., 1999. Protein phosphatase 2A suppresses MAP kinase signaling and ectopic protein expression. Cell Signal. 11, 575-580.
Cummings, J. L., Vinters, H. V., Cole, G. M., Khachaturian, Z. S., 1998. Alzheimer's disease: etiologies, pathophysiology, cognitive reserve, and treatment opportunities. Neurology 51, S2-S17.
Dineley, K/T., Westerman, M., Bui, D., Bell, K., Ashe, K. H., Sweatt, J. D., 2001. Beta-amyloid activates the mitogen-activated protein kinase cascade via hippocampal alpha7 nicotinic acetylcholine receptors: in vitro and in vivo mechanism is related to Alzheimer's disease. J. Neurosci. 21, 4125-4133.
Etcheberrigaray, R., Hirashima, N., Nee, L., Prince, J., Govoni, S., Racchi, M., Tanzi, R. E., Alkon, D. L., 1998. Calcium responses in fibroblasts from asymptomatic members of Alzheimer's disease families. Neuro-biol. Dis. 5, 37-45.
Fernandez, J. J., Candenas, M. L., Souto, M. L., Trujillo, M. M., Norte, M., 2002. Okadaic acid, useful tool for studying cellular processes. Curr. Med. Chem. 9, 229-262.
Ferrell Jr., J. E., 1998. How regulated protein translocation can produce switch-like responses. Trends Biochem. Sc. 23, 461-465.
Force, T., Bonventre, J. V., 1998. Growth factors and mitogen-activated protein kinases. Hypertension 31, 152-161.
Goedert, M., Spillantini, M. G., Cairns, N. J., Crowther, R. A., 1992. Tau proteins of Alzheimer paired helical filaments: abnormal phosphorylation of all six brain isoforms. Neuron 8, 159-168.
Gold, B. G., 1999. FK506 and the role of the immunophilin FKBP-52 in nerve regeneration. Drug Metab. Rev. 31, 649-663.
Gong, C. X., Lidsky, T., Wegiel, J., Zuck, L., Grundke-lqbal, I., Iqbal, K., 2000. Phosphorylation of microtubule-associated protein tau is regulated by protein phosphatase 2A in mammalian brain. Implications for neurofibrillary degeneration in Alzheimer's disease. J. Biol. Chem. 275, 5535-5544.
Gong, C. X., Shaikh, S., Wang, J. Z., Zaidi, T., Grundke-Iqbal, I., Iqbal, K., 1995. Phosphatase activity toward abnormally phosphorylated tau; decrease in Alzheimer disease brain. J. Neurochem. 65, 732-738.
Gonzales, F. A., Seth, A., Raden, D. L., Bowman, D. S., Fay, F. S., Davis, R. J., 1993. Serum-induced translocation of mitogen-activated protein kinase to the cell surface ruffling membrane and the nucleus. J. Cell Biol. 122, 1089-10101.
Guise, S., Braguer, D., Carles, G., Delacourte, A., Briand, C., 2001. Hyperphosphorylation of tau is mediated by ERK activation during anticancer drug-induced apoptosis in neuroblastoma cells. J. Neurosci. Res. 63, 257-267.
Heid, C. A., Stevens, J., Livak, K. J., Williams, P. M., 1996. Real time quantitative PCR. Genome Res. 6, 986-994.
Janssens, V., Goris, J., 2001. Protein phosphatase 2A: a highly regulated family or serine/threonine phosphatases implicated in cell growth and signaling. Biochem. J. 353, 417-439.
Janssens, V., Jordens, J., Stevens, I., Van Hoof, C., Martens, E., de Smedt, H., Engelborghs, Y. M., Waelkens, E., Goris, J., 2003. Identification and functional analysis of two Ca2+-binding EF-hand motifs in the B″/PR72 subunit of protein phosphatase 2A. J. Biol. Chem. 278, 10696-10706.
Jellinger, K. A., Bancher, C., 1998. Neuropathology of Alzheimer's disease: a critical update. J. Neural Transm. Suppl. 54, 77-95.
Kohkhlatchev, A. V., Canagarajah, B., Wilsbacher, J., Robinson, M., Atkinson, M., Goldsmith, E., Cobb, M. H., 1998. Phosphorylation of the MAP kinase ERK2 promotes its homodimerization and nuclear translocation. Cell 93, 605-615.
Kins, S., Crameri, A., Evans, D. R., Hemmings., B. A., Nitsch, R. M., Gotz, J., 2001. Reduced protein phosphatase 2A activity induces hyperhosphorylation and altered compartmentalization of tau in transgenic mice. J. Biol. Chem. 276, 38193-38200.
Klettner, A., Baumgrass, R., Zhang, Y., Fischer, G., Burger, E., Herdegen, T., Mielke, K., 2001. The neuroprotective actions of FK506 binding protein ligands: neuronal survival is triggered by de novo RNA synthesis, but is independent of inhibition of NJK and calcineurin. Brain Res. Mol. Brain Res. 97, 21-31.
Knowles, R. B., Chin, J., Ruff, C. T., Hyman, B. T., 1999. Demonstration by fluorescence resonance energy transfer of a close association between activated MAP kinase and neurofibrillary tangles: implications for MAP kinase activation in Alzheimer disease. J. Neuropathol. Exp. Neurol. 58, 1090-1098.
Lee, V. M., 1995. Disruption of the cytoskeleton in Alzheimer's disease. Curr. Opin. Neurobiol. 5, 663-668.
Lenormand, P., Sardet, C., Pages, G., L'Allemain, G., Brunet, A., Pouyssegur, J., 1993. Growth factors induce nuclear translocation of MAP kinase (p42mapk and /44mapk) but not of their activator MAP kinase (p45mapkk) in fibroblasts. J. Cell Biol. 122, 1079-1088.
Lewis, T. S., Shapiro, P. S., Ahn, N. G., 1998. Signal transduction through MAP kinase cascades. Adv. Cancer Res. 74, 49-139.
Liebmann, C., 2001. Bradykinin signaling to MAP kinase: cell specific connections versus principle mitogenic pathways. Biol. Chem. 382, 49-55.
Livak, K. J., Schimttgen, T. D., 2001. Analysis of relative gene expression data using real-time quantitative PCR and the 2^{-AAC}_{T} method. Methods. 25, 402-408.
Mandelkow, E., Song, Y. H., Schweers, O., Marx, A., Mandelkow, E. M., 1995. On the structure of microtubules, tau, and paired helical filaments. Neurobiol. Aging 16, 347-354.
Matsubayashi, Y., Fukuda, M., Nishida, E., 2001. Evidence for existence of a nuclear pore complex-mediated, cytosol-independent pathway of nuclear translocation of ERK MAP kinase in permeabilized cells. J. Biol. Chem. 276, 41755-41760.
Mattson, M., Chan, S. L., Camandola, S., 2001. Presenilin mutations and calcium signaling defects in the nervous and immune systems. BioEssays 23.8, 733-744.
McMahon, K. A., Wilson, N. J., Marks, D. C., Beecroft, T. L., Whitty, G. A., Hamilton, J. A., Csar, X. F., 2001. Colony-stimulating factor-1 (CSF-1) receptor-mediated macrophase differentiation in myeloid cells: a role for tyrosine 559-dependent protein phosphatase 2A (PP2A) activity. Biochem. J. 358, 431-436.
Nagao, M., Shima, H., Nakayasu, M., Sugimura, T., 1995. Protein serine/threonine phosphatase as binding proteins for okadaic acid. Mutat. Res. 333, 173-179.
Planel, E., Yasutake, K., Fujita, S. C., Ishiguro, K., 2001. Inhibition of protein phosphatase 2A overrides tau protein kinase l/glycogen synthase kinase 3, and cyclin-dependent kinase 5 inhibition and results in tau hyperphosphorylation in the hippocampus of starved mouse. J. Biol. Chem. 276, 34289-34306.
Putney Jr., J. W., 2000. Presenilins, Alzheimer's disease, and capacitative calcium entry. Neuron 27, 411-412.
Rapport, M., Ferreira, A., 2000. PD 98059 prevents neurite degeneration induced by fibrillar beta-amyloid in mature hippocampal neurons. J. Neurochem. 74, 125-133.
Roovers, K., Assoian, R. K., 2000. Integrating the MAP kinase signal into the GI phase cell cycle machinery. Bioessays 22, 818-826.
Saito, T., Ishiguro, K., Uchida, T., Miyamoto, E., Kishimoto, T., Hisanaga, S., 1995. In situ dephosphorylation of tau by protein phosphatase 2A and 2B in fetal rat primary cultured neurons. FEBS Lett. 376, 238-242.
Sheehan, J. P., Swerdlow, R. H., Miller, S. W., Davis, R. E., Parks, J. K., Parker, W. D., Tuttle, J. B., 1997. Calcium homeostasis and reactive oxygen species production in cells transformed by mitochondria from individuals with sporadic Alzheimer's disease. J. Neurosci. 17, 4612-4622.
Sheppeck, J. E., Gauss, C. M., Chamberlin, A. R., 1997. Inhibition of the Ser-Thr phosphatases PP1 and PP2A by naturally occurring toxins. Bioorg. Med. Chem. 5, 1739-1750.
Silverstein, A. M., Barrow, C. A., Davis, A. J., Mumby, M. C., 2002. Actions of PP2A on the MAP kinase pathway and apoptosis are mediated by distinct regulatory subunits. Proc. Natl. Acad. Sci. USA 99, 4421-4426.
Sweatt, J. D., 2001. The neuronal MAP kinase cascade: a biochemical signal integration system subserving synaptic plasticity and memory. J. Neurochem. 76, 1-10.
Treisman, R., 1996. Regulation of transcription by MAP kinase cascades. Curr. Opin. Cell Biol. 8, 205-215.
Valjent, E., Caboche, J., Vanhoutte, P., 2001. Mitogen-activated protein kinase/extracellular signal-regulated kinase induced gene regulation in brain: a molecular substrate for learning and memory. Mol. Neurobiol. 23, 83-99.
Vogelsberg-Ragaglia, V., Schuck, T., Trojanowski, J. Q., Lee, V. M., 2001. PP2A mRNA expression is quantitatively decreased in Alzheimer's disease hippocampus. Exp. Neurol. 168, 402-412.
Volmat, V., Camps, M., Arkinstall, S., Pouyssegur, J., Lenormand, P., 2001. The nucleus, a site for signal termination by sequestration and inactivation of p42/p44 MAP kinases. J. Cell Sci. 114, 3433-3443.
Wang, J. Z., gong, C. X., Zaidi, T., Grundke-Iqbal, I., Iqbal, K., 1995. Dephosphorylation of Alzheimer paired helical filaments by protein phosphatase-2A and -2B. J. Biol. Chem. 270, 4854-4860.
Winer, J., Jung, C. K., Shackel, I., Williams, P. M., 1999. Development and validation of real-time quantitative reverse transcriptase-polymerase chain reaction for monitoring gene expression in cardiac myocytes in vitro. Anal. Biochem. 270, 41-49.
Winter, C., Schenkel, J., Zimmermann, M., Herdegen, T., 1998. MAP kinase phosphatase 1 is expressed and enhanced by FK506 in surviving mamillary, but not degenerating nigral neurons following axotomy. Brain Res. 801, 198-205.
Yoo, A. S., Cheng, I., Chung, S., Grenfell, T. Z., Lee, H., Pack-Chung, E., Handler, M., Shen, J., Xia, W., Tesco, G., Saunders, A. J., Ding, K., Frosch, M. P., Tanzi, R. E., Kim, T. W., 2000. Presenilin-mediated modulation of capacitative calcium entry. Neuron 27, 561-572.
Zawadzka, M., Kaminska, B., 2003. Immunosuppressant FK506 affects multiple signaling pathways and modulates gene expression in astrocytes. Mol. Cell. Neurosci. 22, 202-209.
Zhao, W. Q., Chen, H., Xu, H., Moore, E., Merri, N., Quon, M. J., Alkon, D. L., 1999. Brain insulin receptors and spatial memory. Correlated changes in gene expression, tyrosine phosphorylation, and signaling molecules in the hippocampus of water maze trained rats. J. Biol. Chem. 274, 34893-34902.
Zhao, W. Q., Ravindranath, L., Mohamed, A. S., Zohar, O., Chen, G. H., Lyketsos, C. G., Etcheberrigaray, R., Alkon, D. L., 2002. MAP kinase signaling cascade dysfunction specific to Alzheimer's disease in fibroblasts. Neurobiol. Dis. 11, 166-183.
Zhao, W. Q., Feng, C., Alkon, D. L. 2003. Impairment of phosphatase 2A contributes to the prolonged MAP kinase phosphorylation in Alzheimer's disease fibroblasts. Neurobiol. Dis. 14, 458-469.

What is claimed is:

1. A method of diagnosing Alzheimer's disease in a subject, said method comprising the steps of:
a. obtaining a cell sample from said subject; and
b. detecting the level of PP2A gene expression in said sample, wherein an elevated level of PP2A gene expression compared to control cells indicates the presence of Alzheimer's disease.

2. The method of claim 1, wherein said cell sample is selected from the group consisting of fibroblasts, buccal mucosal cells, neurons, and blood cells.

3. The method of claim 1, wherein said cells are fibroblasts.

4. The method of claim 1, wherein the detecting step (b) is performed by reverse transcription quantitative polymerase chain reaction (RVQ-PCR).

5. A method of diagnosing Alzheimer's disease in a subject, said method comprising the steps of:
a. obtaining a cell sample from said subject;
b. contacting said cell sample with an agent that stimulates phosphorylation of a PP2A substrate to stimulate the cells; and
c. comparing the level of PP2A gene expression in said stimulated cells to the level of PP2A gene expression in unstimulated cells of the same type from said subject, wherein a lack of increased PP2A gene expression in stimulated cells as compared to unstimulated cells indicates the presence of Alzheimer's disease.

6. The method of claim 5, wherein said agent is bradykinin.

7. The method of claim 5, wherein said PP2A substrate is Erk1/2.

8. The method of claim 5, wherein said cells are selected from the group consisting of fibroblasts, buccal mucosal cells, neurons, and blood cells.

9. The method of claim 5, wherein said cells are fibroblasts.

10. The method of claim 5, wherein the comparing step (c) is performed by calculating a ratio of PP2A gene expression in the presence and absence of the agent that stimulates phosphorylation of a PP2A substrate.

11. A method of diagnosing Alzheimer's disease in a subject, said method comprising the steps of:
a. obtaining a cell sample from said subject;
b. detecting the level of PP2A protein or enzymatic activity in said cell sample, wherein a reduced level of PP2A protein or enzymatic activity compared to non-Alzheimer's control cells indicates the presence of Alzheimer's disease.

12. The method of claim 11, wherein said cell sample is selected from the group consisting of fibroblasts, buccal mucosal cells, neurons, and blood cells.

13. The method of claim 11, wherein said cells are fibroblasts.

14. The method of claim 11, wherein detecting the level of PP2A protein is performed by Western blot or ELISA.

15. A method of diagnosing Alzheimer's disease in a subject, said method comprising the steps of:
a. obtaining a cell sample from a subject;
b. contacting said cell sample and control cells with a first agent that stimulates phosphorylation of a substrate of PP2A and, a second agent that is an inhibitor of PP2A;
c. measuring the level of phosphorylation of the PP2A substrate at a predetermined time after initiating the contacting step; and
d. comparing the level of phosphorylation of the PP2A substrate in said cell sample to the level of PP2A substrate phosphorylation in control cells at the same predetermined time, wherein a lack of additional effect of the PP2A inhibitor on the extent of the PP2A substrate phosphorylation in the cell sample compared to control cells indicates the presence of Alzheimer's disease.

16. The method of claim 15, wherein the PP2A substrate is Erk1/2.

17. The method of claim 15, wherein the inhibitor of PP2A is okadiac acid.

18. The method of claim 15, wherein the cells are selected from the group of fibroblasts, buccal mucosal cells, neurons, and blood cells.

19. The method of claim 15, wherein the cells are fibroblasts.

20. The method of claim 15, wherein said agent that stimulates phosphorylation is bradykinin.

21. The method of claim 15, wherein the comparing step (d) is performed by calculating a test ratio of PP2A substrate phosphorylation in the presence and absence of the PP2A inhibitor, wherein said test ratio is significantly greater in control cells than in Alzheimer's disease cells.

22. A method of diagnosing Alzheimer's disease in a subject, said method comprising the steps of:
a. obtaining a cell sample from a subject;
b. contacting control cells and said cell sample with a first agent that stimulates phosphorylation of a substrate of PP2A, wherein said contacting is done in the presence and the absence of a second agent that is an inhibitor of PP2A;
c. measuring the level of phosphorylation of the PP2A substrate from said control cells and said cell sample at a predetermined time after initiating the contacting step (b); and
d. comparing the level of phosphorylation of the PP2A substrate from said cell sample in the presence and the absence of said second agent that is an inhibitor of PP2A, wherein a lack of a significant difference between the extent of PP2A substrate phosphorylation in the presence and the absence of said second agent indicates the presence of Alzheimer's disease.

23. The method of claim 22, wherein said control cells show a significant difference in the level of phosphorylation of the PP2A substrate in the presence and the absence of said second agent that is an inhibitor of PP2A.

24. The method of claim 22, wherein said cell sample is selected from the group consisting of fibroblasts, buccal mucosal cells, neurons, and blood cells.

25. The method of claim 22, wherein said cell sample is fibroblasts.

26. The method of claim 22, wherein said first agent that stimulates phosphorylation of a PP2A substrate is bradykinin.

27. The method of claim 22, wherein said second agent that is an inhibitor of PP2A is okadiac acid.

28. The method of claim 22, wherein said PP2A substrate is Erk1/2.

29. A method of diagnosing Alzheimer's disease in a subject, said method comprising the steps of:
a. obtaining a cell sample from said subject; and
b. contacting said sample with an agent that stimulates phosphorylation of Erk1/2; and
c. detecting the subcellular distribution of phosphorylated Erk1/2, wherein an extranuclear distribution of phosphorylated Erk1/2 indicates the presence of Alzheimer's disease.

30. The method of claim 29, wherein the compound that stimulates phosphorylation of Erk1/2 is bradykinin.

31. The method of claim 29, wherein the detecting step (c) is performed by immunocytochemistry or by determining a test ratio of phosphorylated Erk1/2 between the nucleus and the cytosol of the sample cells.

32. A method of diagnosing Alzheimer's disease in a subject comprising any combination of the diagnosis methods of claims 1, 5, 11, 15, 22 and 29.

33. A method of diagnosing Alzheimer's disease in a subject comprising any combination of the diagnosis methods of claims 1, 5, 11, 15, 22 and 29, in further combination with methods of diagnosing Alzhiemer's disease based on measuring increased phosphorylation of a MAPK protein after stimulation with an agent that triggers intracellular calcium release.

34. A method of screening to identify a substance useful for treatment or prevention of Alzheimer's disease comprising the steps of:
a. contacting a cell sample with the substance being tested;
b. determining whether the substance reverses or improves PP2A Alzheimer's disease-associated abnormalities, wherein a compound that reverses or improves said PP2A abnormalities is identified as a therapeutic substance useful for the treatment or prevention of Alzheimer's disease.

35. The method of claim 34, wherein said Alzheimer's disease-associated abnormality is the presence of increased PP2A mRNA compared to non-Alzheimer's control cells.

36. The method of claim 34, wherein said Alzheimer's disease-associated abnormality is the lack of increased PP2A expression in cells contacted with an agent that stimulates phosphorylation of Erk1/2.

37. The method of claim 34, wherein said Alzheimer's disease-associated abnormality is reduced PP2A protein or PP2A enzymatic activity compared to non-Alzheimer's control cells.

38. The method of claim 34, wherein said Alzheimer's disease-associated abnormality is the lack of a normal response when test cells are treated with bradykinin in the presence of okadiac acid.

39. The method of claim 34, wherein said Alzheimer's disease-associated abnormality is distribution of phosphorylated Erk1/2 in the extranuclear area.

40. A diagnostic test kit for Alzheimer's disease comprising bradykinin and oligonucleotide PCR primers specific for a nucleic acid sequence encoding a PP2A protein.

41. A diagnostic test kit for Alzheimer's disease comprising an anti-PP2A antibody.

42. A diagnostic test kit for Alzheimer's disease comprising an anti-Erk1/2 antibody and bradykinin.

43. A diagnostic test kit for Alzheimer's disease comprising an anti-phospho Erk1/2 antibody and bradykinin.

44. A diagnostic test kit for Alzheimer's disease comprising bradykinin, okadiac acid and an anti-Erk1/2 antibody.

45. The diagnostic test kit of claim 44, further comprising an anti-phospho Erk1/2 antibody.
